(19) United States
(12) Patent Application Publication
(10) Pub. No.: US 2024/0166993 A1
(43) Pub. Date: May 23, 2024

(54) METHOD FOR SEPARATING SPERMATOGONIAL STEM CELLS (SSCs) FROM FROZEN TESTICULAR TISSUE

(71) Applicant: Northwest Institute of Plateau Biology, CAS, Xining City (CN)

(72) Inventors: Qi'en Yang, Xining City (CN); Ruidong Wan, Xining City (CN); Gongxue Jia, Xining City (CN); Shixin Wu, Xining City (CN); Yougui Fang, Xining City (CN)

(21) Appl. No.: 18/451,063

(22) Filed: Aug. 16, 2023

(30) Foreign Application Priority Data
Nov. 18, 2022 (CN) 202211442996.2

(51) Int. Cl. C12N 5/071; C12N 9/22; C12N 9/64

(52) U.S. Cl. CPC C12N 5/0612; C12N 9/22; C12N 9/6489

(57) ABSTRACT

The present disclosure provides a method for separating SSCs from a frozen testicular tissue, and belongs to the field of cell biology. In the present disclosure, the method includes the following steps: resuscitating a frozen testicular tissue, digesting the resuscitated testicular tissue with a collagenase IV and a DNase I until seminiferous tubules are exposed and tube walls become coarse; washing the digested testicular tissue with a DPBS, conducting centrifugation and culture until cells migrate; collecting the migrated cells, and enriching and purifying SSCs. The method can maintain a milieu interieur of the SSCs to the greatest extent, and therefore cell viability, and less cell damages. Therefore, the method can be used for the separation of SSCs from frozen testicular tissues.

### CROSS REFERENCE TO RELATED APPLICATION

This patent application claims the benefit and priority of Chinese Patent Application No. 202211442996.2 filed with the China National Intellectual Property Administration on Nov. 18. 2022, the disclosure of which is incorporated by reference herein in its entirety as part of the present application.

### TECHNICAL FIELD

The present disclosure belongs to the field of cell biology, and in particular relates to a method for separating SSCs from a frozen testicular tissue.

### BACKGROUND

Spermatogenesis includes three stages: spermatogonial mitosis, spermatocyte meiosis, and spermiogenesis. The mitosis forms undifferentiated spermatogonia (such as A single type (Aₛ), A paired type (Aₚᵣ), and A aligned type (Aₐₗ)), and differentiated spermatogonia (such as A₁, A₂, A₃, A₄, In, and B types). The undifferentiated spermatogonia include SSCs capable of maintaining a balance between self-renewal of the stem cell pool and continuous sperm production. The SSCs play an important role in maintaining spermatogenesis. As the only adult stem cells that provide genetic information to the next generation, SSCs have broad application prospects in the fields of animal genetics, breeding, and reproduction. In 1994, Brinster et al. microinjected SSCs from a donor mouse into the testes of a recipient mouse that was treated with busulfan in advance. The SSCs colonized in seminiferous tubules and restarted spermatogenesis to produce offspring. This experiment has pioneered the allogeneic transplantation technique of SSCs. Through gene transfection and homologous transplantation of SSCs, not only the excellent traits of elite breeding stock can be inherited, but also transgenic animals with commercial values can be produced, thereby accelerating the breeding.

However, SSCs show an extremely low proportion in the testis, with only 0.2% to 0.3% in bovine testis. Therefore, it is necessary to conduct enrichment and purification on separated SSCs to meet the needs of culture and transplantation. The separation of SSCs mainly adopts a two-step enzyme digestion method. The enrichment and purification of SSCs mainly rely on magnetic activated cell sorting (MACS), fluorescence-activated cell sorting (FACS), differential adhesion, and density gradient centrifugation. Compared with other enrichment methods, the differential adhesion has the best effect on the enrichment of SSCs, but shows lower specificity. The MACS and FACS can enrich high-purity SSCs. For example, Li et al. obtained Thy 1⁺ SSCs population by the MACS and FACS, but the aforementioned methods are limited by specific marker genes on the cell surface. The density gradient centrifugation is widely used in the enrichment and purification of SSCs from animals such as human beings, monkeys, sheep, pigs, and cattle. In the enrichment and purification of bovine SSCs, Izadyar et al. purified a testicular cell suspension containing 25.5% bovine type A spermatogonia to 51% by Percoll gradient centrifugation: and Oatley et al. separated a cell fraction of bovine that was highly enriched with SCCs by Percoll gradient centrifugation and differential adhesion. However, trypsin in the two-step enzyme digestion method has relatively large damages to the cells, and can cause certain adverse effects to cell viability: Especially when a tissue to be separated is a frozen tissue, since the frozen tissue is more fragile than a fresh one, it is difficult to effectively separate SSCs with high viability. Accordingly, it is a key to research in this field to find a method suitable for separating SSCs from frozen tissues with reduced cell damages during the process.

### SUMMARY

In view of this, an objective of the present disclosure is to provide a method for separating SSCs from a frozen testicular tissue, while maintaining a milieu interieur of the SSCs to the greatest extent, and therefore cell viability and less cell damages.

To achieve the above objective, the present disclosure provides the following technical solutions:

The present disclosure provides a method for separating SSCs from a frozen testicular tissue, including the following steps:
resuscitating a frozen testicular tissue, digesting the resuscitated testicular tissue with a collagenase IV and a DNase I until seminiferous tubules are exposed and tube walls become coarse: washing the digested testicular tissue with a Dulbecco's phosphate-buffered saline (DPBS), conducting centrifugation, and culture until cells migrate: collecting the migrated cells, and enriching and purifying SSCs.

Preferably, the resuscitating includes: resuscitating a cryopreservation tube in a water bath at 36° ° C. to 38° C. until the cryopreservation tube is completely thawed.

In some embodiments, the frozen testicular tissue includes a frozen bovine testicular tissue.

In some embodiments, the resuscitated testicular tissue is digested by sequentially adding the DPBS, a collagenase IV solution, and a DNase I solution that are in an equal volume.

In some embodiments, the collagenase IV solution has a concentration of 0.5 mg/mL to 2 mg/mL, and the DNase I solution has a concentration of 0.5 mg/mL to 2 mg/mL.

In some embodiments, the digesting is conducted in a water bath at 36° ° C. to 38° C. for incubation.

In some embodiments, the culture is conducted with a DFS (Components include Dulbecco's Modified Eagle Medium (DMEM), Fetal Bovine Serum (FBS), Penicillin/Streptomycin (P/S) Solution) medium in an incubator at 36° ° C. to 38° C. with 5% CO2.

In some embodiments, enriching and purifying SSCs includes: conducting Percoll gradient centrifugation and purification in sequence to obtain the SSCs.

In some embodiments, the Percoll gradient centrifugation includes: adding a 40% Percoll solution, a 20% Percoll solution, and a suspension of the migrated cells sequentially into a centrifuge tube, conducting centrifugation, and collecting an interphase layer between the 40% Percoll solution and the 20% Percoll solution to obtain the SSCs.

In some embodiments, the 40% Percoll solution, the 20% Percoll solution, and the suspension of the migrated cells are at a volume ratio of 1:1:2.

Compared with the prior art, embodiments of the present disclosure have the following beneficial effects:

In the present disclosure, temporary culture of the seminiferous tubules stabilizes the milieu interieur of SSCs allows the cells to migrate naturally, and reduces the damage to the cells by trypsin in the two-step enzyme digestion method, thus maintaining cell viability. The method is more suitable for separation of SSCs in frozen testicular tissues. The method not only solves the problem that tissues for separating cells are not long-term preserved, but also provides important support for the preservation of genetic information of rare and endangered species, and the restoration and updating of individuals.

In the present disclosure, the method is simple, convenient, and easy to operate, which is conductive to promoting the implementation.

### BRIEF DESCRIPTION OF THE DRAWINGS

FIG. 1 shows exposed seminiferous tubules after enzymatic digestion:

FIG. 2 shows migration and adhesion of cells in the seminiferous tubules:

FIG. 3 shows cluster-like growth of SSCs, where black arrows indicate the SSCs:

FIG. 4 shows identification of the SSCs by fluorescent staining.

### DETAILED DESCRIPTION OF THE EMBODIMENTS

The present disclosure provides a method for separating SSCs from a frozen testicular tissue, including the following steps:
resuscitating a frozen testicular tissue, digesting the resuscitated testicular tissue with a collagenase IV and a DNase I until seminiferous tubules are exposed and tube walls become coarse: washing the digested testicular tissue with a DPBS, conducting centrifugation and culture until cells migrate: collecting the migrated cells, and enriching and purifying SSCs.

In the present disclosure, the frozen testicular tissue is preferably a frozen bovine testicular tissue.

In the present disclosure, the frozen testicular tissue is subjected to resuscitation before the separation of SSCs. The resuscitation includes: taking a cryopreservation tube out of liquid nitrogen, immediately placing in a water bath at 36° ° C. to 38° C. for resuscitation, followed by subsequent cell separation experiments after complete thawing. The water bath is more preferably at 37° C.

In the present disclosure, the seminiferous tubules in the resuscitated testicular tissue are first separated: before the separation, a thawed testis tissue is preferably washed with DPBS. As an optional embodiment, the thawed testicular tissue is put into a centrifuge tube, DPBS containing 10% P/S solution was added, a resulting mixture was allowed to stand on ice for 4 min to 6 min, and a supernatant was removed. The above processes are repeated 2 to 3 times.

In the present disclosure, the resuscitated testicular tissue is digested by adding the DPBS, a collagenase IV solution, and a DNase I solution in sequence: the DPBS, the collagenase IV solution, and the DNase I solution are at a volume ratio of preferably 1:1:1. The collagenase IV solution has a concentration of 0.5 mg/mL to 2 mg/mL, preferably 1 mg/mL: and the DNase I solution has a concentration of 0.5 mg/mL to 2 mg/mL, preferably 1 mg/mL. The digestion is conducted in a water bath at 36° ° C. to 38° C. for incubation. As an optional embodiment, a pre-digestion system is incubated in a 37° ° C. water bath, shaken several times during the incubation, and then observed under a microscope until the seminiferous tubules are exposed and tube walls become coarse. At this time, the digestion is terminated.

In the present disclosure, the DPBS is added to the separated seminiferous tubules to terminate the digestion, the digested seminiferous tubules are allowed to stand on ice for 8 min to 15 min, and a resulting supernatant is discarded. The above operations are repeated 2 to 3 times to remove interstitial cells. A resulting pellet is resuspended with DPBS and centrifuged, and cells are resuspended with a DFS medium, and then cultured until the cells migrate. The culture is conducted at 36° ° C. to 38° C. , preferably 37° C. , for preferably 22 h to 24 h. As an optional embodiment, the cells are resuspended in a DFS medium and inoculated into a 6-well plate, and then cultured in an incubator with 5% CO₂.

In the present disclosure, the DFS medium includes preferably the following components: 88.5% to 90% of a DMEM . 8.5% to 10.5% of FBS. and 1% to 1.5% of a P/S solution.

In the present disclosure, the plate inoculated with the seminiferous tubules is taken out. and the migration of cells is observed under a microscope. After observation of the cell migration. the cells are pipetted with a pipette, and a cell suspension is collected and allowed to stand for 8 min to 12 min. An obtained supernatant is passed through a 40-μm cell sieve, and the sieved cells are collected.

In the present disclosure, the collected cells are washed to prepare a DPBS cell suspension. As an optional embodiment, the sieved cells are centrifuged to remove a supernatant, washed with the DPBS, centrifuged again to remove a supernatant, and resuspended with the DPBS to obtain the DPBS cell suspension. The centrifugation is preferably conducted at 600 g for 5 min.

In the present disclosure, a process of the enrichment and purification of the SSCs includes: conducting Percoll gradient centrifugation and purification in sequence to obtain the SSCs.

In the present disclosure. Percoll gradient centrifugation is conducted on the DPBS cell suspension. The Percoll solution includes a 20% Percoll solution and a 40% Percoll solution. In the present disclosure, the 40% Percoll solution. the 20% Percoll solution, and a suspension of the migrated cells are sequentially added in the centrifuge tube: the 40% Percoll solution, the 20% Percoll solution, and the suspension of the migrated cells are at a volume ratio of 1:1:2. After the centrifugation, an interphase layer between the 40% Percoll solution and the 20% Percoll solution is collected (after the centrifugation. due to different densities, there is stratification, and SSCs are located in the interphase layer of the 40% Percoll solution and the 20% Percoll solution), to obtain the SSCs. The centrifugation is optionally conducted at 600 g for 7 min.

In the present disclosure, a method for preparing the 40% Percoll solution and 20% Percoll solution includes:
40% Percoll solution: adding 10% of a 10xphosphate-buffered saline (PBS), 50% of ultrapure water, and 40% of a Percoll solution to 20 uL of a phenol red solution, and adjusting a pH value of a resulting mixture to 7.0 with concentrated hydrochloric acid.
20% Percoll solution: adding 10% of a 10×PBS. 70% of ultrapure water, and 20% of a Percoll solution to 20 uL of a phenol red solution. and adjusting a pH value of a resulting mixture to 7.0 with concentrated hydrochloric acid.

In the present disclosure, the mesophase of the 40% Percoll solution and the 20% Percoll solution is preferably washed 2 to 3 times with DPBS, and then resuspended with an SSCs medium to obtain the cell suspension. As an optional embodiment, after the addition of DPBS, the mesophase is pipetted evenly and centrifuged, an obtained supernatant is discarded, and the DPBS washing is repeated: the centrifugation is optionally conducted at 600 g for 5 min.

In the present disclosure, an obtained SSCs medium suspension is inoculated into a culture plate covered with trophoblast cells for culture. Preferably, the cells are cultured in an incubator. where a culture medium is changed every 2 d to 3 d, and subculture is conducted for 5 d to 7 d.

As an optional embodiment. a method for preparing the trophoblast cells includes: culturing mouse embryonic fibroblasts (MEFs) with a DFS medium until the confluence reaches 70% to 80%, treating the MEFs with mitomycin C (10 ng/ml) for 2 h, digesting the cells with 0.25% trypsin for 3 min, adding DFS medium to terminate the digestion, collecting a resulting cells by centrifugation at 600 g for 5 min. resuspending the MEFs with DFS medium, counting cells, and inoculating the MEFs into a 6-well plate at a concentration of 5×10⁵ cells/mL.

In the present disclosure, the separated SSCs can be subcultured. A subculture method may optionally include: discarding the culture medium by aspirating when the confluence in the culture plate reaches 70% to 80%, adding 0.25% trypsin for digestion, adding DFS medium to terminate the digestion after about 3 min, pipetting the cells into a centrifuge tube, which is then centrifuged at 600 g for 5 min, discarding a resulting supernatant, resuspending the obtained cells in SSCs medium, and then inoculating the cells into a culture plate previously covered with trophoblast cells for culture.

The technical solutions provided by the present disclosure will be described in detail below with reference to examples, but the examples should not be construed as limiting the claimed scope of the present disclosure.

In the examples of the present disclosure:
DFS medium: 89% DMEM+10% FBS+1% P/S solution:
40% Percoll solution: 10% of a 10×PBS. 50% of ultrapure water, and 40% of a Percoll solution are added to 20 uL of a phenol red solution, and a pH value of a resulting mixture is adjusted to 7.0 with concentrated hydrochloric acid:
20% Percoll solution: 10% of a 10×PBS, 70% of ultrapure water, and 20% of a Percoll solution are added to 20 uL of a phenol red solution, and a pH value of a resulting mixture is adjusted to 7.0 with concentrated hydrochloric acid:
preparation of trophoblast cells: mouse embryonic fibroblasts (MEFs) are cultured with the DFS medium. When the confluence reaches 70% to 80%, the MEFs are treated with mitomycin C (10 ng/ml) for 2 h. After the cells are digested with 2 mL of 0.25% trypsin for 3 min, 2 mL of the DFS medium is added to terminate the digestion. The resulting cells are collected by centrifugation at 600 g for 5 min, the MEFs are resuspended with 1 mL DFS medium, and cell counting is conducted. The MEFs are inoculated into a 6-well plate at a concentration of 5× 10⁵ cells/mL:
SSCs medium: MEM-Alpha basal medium (Gibco, 12561-049), bovine serum albumin (sigma, A6003) 5 mg/mL, holotransferrin (sigma, T0665) 100 μg/mL, insulin (sigma, 12643) 25 ug/mL, L-glutamine solution 100× (Procell, PB180419) 1×, putrescine (sigma, P5780) 20 μg/mL, sodium selenite (sigma, 214485) 0.3 nM, 2-mercaptoethanol (Gibco, 21985) 55 μM, MEM non-essential amino acid 100× solution (Gibco, 11140) 1×, MEM vitamins 100× solution (Gibco, 11120052) 1×, StemPro™ additive 50× solution (Gibco, A1050801) 1×, GDNF (Peprotech, AF-450-10) 20 ng/ml, and FGF (Peprotech, AF-100-18B) 5 ng/ml.

In the following examples, all methods are conventional methods, unless otherwise specified.

All materials and reagents used in the following examples are commercially available, unless otherwise specified.

### Example 1

In this example, a method for separation and culture of SSCs from a bovine testicular tissue under cryopreservation was provided:

### (1) Cryopreservation of Testicular Tissue

Preparation of a cryopreservation solution:

DMEM, FBS, and DMSO were formulated into the cryopreservation solution in a ratio of 7:2:1, and then filtered with a 0.22 μm filter for future use.

Collection and cryopreservation of testicular tissue:

A testicular tissue at the age of 3 to 5 months was collected, the tunica albuginea was removed, and a middle part of the testis was cut across. Thin slices of the testicular tissue (in a soybean size) were slowly scraped with a blade along the cross section, placed into a cryopreservation tube, and then added with about 1.5 mL of the cryopreservation solution to mix evenly: By a gradient cooling method, the cryopreservation tube was placed in parallel at 4° C. for 1 h to 2 h, at −20° ° C. for 1 h to 2 h, and at −80° ° C. for 12 h to 16 h, and finally stored in liquid nitrogen for long time preservation.

### (2) Resuscitation of Frozen Tissue

The cryopreservation tube containing the testicular tissue preserved in liquid nitrogen was taken out, and immediately thawed in a 37° C. water bath, and after complete thawing, the testicular tissue in the cryopreservation tube was transferred to a 50 mL centrifuge tube.

### (3) Separation and Culture of SSCs

Separation and culture of seminiferous tubules:

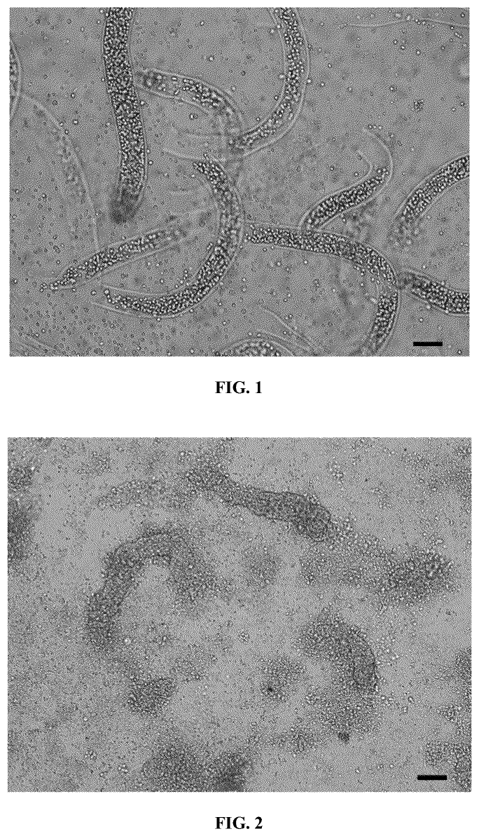

The testicular tissue was washed with DPBS, where 10 mL of DPBS (containing 10% P/S) was added for 3 times of washing, during which the tissue inside the DPBS was dispersed as much as possible by pipetting several times. 5 mL of the DPBS. 5 mL of a collagenase IV solution (1 mg/mL), and 5 mL of a DNase I solution (1 mg/mL) were added to the testicular tissue, and the resulting mixture was digested in a water bath at 37° C. for 15 min, during which the mixture was pipetted several times to disperse the tissue completely, until seminiferous tubules were exposed and tube walls became coarse (FIG. 1). The digestion was terminated with 30 mL of DPBS, the centrifuge tube was placed on ice to conduct sedimentation for 10 min, and an obtained supernatant was removed. The remaining testicular tissue was washed with 10 mL of DPBS. The above steps were repeated 3 times to remove the interstitial cells as much as possible while retaining the seminiferous tubules. Centrifugation was conducted at 400 g for 5 min, and an obtained supernatant was discarded. The seminiferous tubules were resuspended with 3 mL of DFS medium, inoculated into a 6-well plate, and then cultured in a cell incubator at 37° ° C. with 5% CO₂ for 24 h. Enrichment and purification of SSCs:

The culture plate was taken out after 24 h when the seminiferous tubules adhered to the wall, and a large number of cells in the tubes migrated (FIG. 2). The cells were pipetted with a pipette, a cell suspension was collected and allowed to stand for 10 min, and an obtained supernatant was passed through a 40 um cell sieve. The sieved cells were collected together. centrifuged at 600 g for 5 min. and a resulting supernatant was removed: the remaining cells were washed with DPBS, centrifuged at 600 g for 5 min. and then resuspended with 2 mL of DPBS. 1 mL of a 40% Percoll solution, 1 mL of a 20% Percoll solution, and 2 mL of a resulting cell suspension were added to a 15 mL centrifuge tube in sequence. A resulting mixture was centrifuged at 600 g for 7 min, and an interphase layer between the 40% Percoll solution and the 20% Percoll solution was added to 8 mL of DPBS to mix evenly by pipetting, and then centrifuged at 600 g for 5 min. An obtained supernatant was removed, and the above steps were repeated twice. A resulting product was resuspended in 3 mL of an SSCs medium, then inoculated into a culture plate covered with trophoblast cells, and cultured in an incubator, where the culture medium was changed every 2 d to 3 d, and subculture was conducted for 5 d to 7 d.

### (4) Passage of SSCs

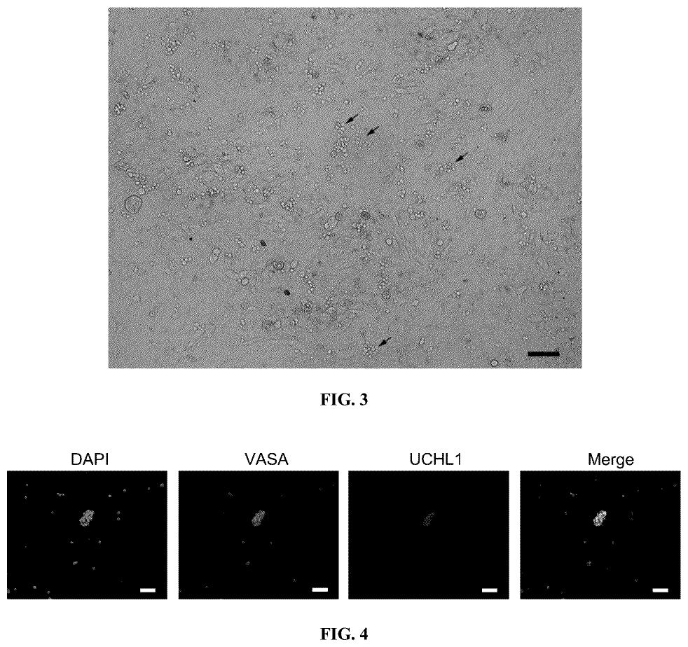

When the confluence in the culture plate reached 70% to 80%, the culture medium was discarded by aspirating. 2 mL of 0.25% trypsin was added for digestion. When the cells were digested and rounded. 2 mL of the DFS medium was added to terminate the digestion. The cells were pipetted into a 15 mL centrifuge tube, centrifuged at 600 g for 5 min, and a resulting supernatant was discarded. The obtained cells are resuspended in the SSCs medium, and then inoculated into a culture plate previously covered with trophoblast cells for culture. At generation P1, the SSCs began to grow in clusters (FIG. 3).

### (5) Identification of SSCs

After taken out, the culture plate was carefully and slowly pipetted to make some cells fall off. The medium was aspirated, and transferred to a 15 mL centrifuge tube, and a new SSCs medium was added to the culture plate. The detached cells were centrifuged at 600 g for 5 min, and an obtained supernatant was discarded. The remaining cells were resuspended in DPBS and centrifuged at 600 g for 5 min, and an obtained supernatant was discarded. A resulting pellet was resuspended in a 4% paraformaldehyde fixative solution, transferred to an adhesive glass slide, and allowed to stand on ice for 20 min. When the cells settled on a surface of the adhesive glass slide, the fixing solution was discarded, DPBS was added, and washing was repeated 3 times by shaking slowly on a shaker, for 5 min each time. A 0.1% Triton X-100 solution was added for permeabilization for 20 min and then discarded by aspiration. The cells were washed 3 times with DPBS, with 5 min for each time. The DPBS was discarded by aspiration, and the cells were blocked with 10% donkey serum for 1 h. A diluted primary antibody, i.e. mouse UCHLI and rabbit VASA, was added to conduct incubation at 4° C. for 16 h. A resulting product was washed 3 times with DPBS, 10 min for each time. A secondary antibody, i.e. donkey anti-rabbit 488 and donkey anti-mouse 594, was added to conduct incubation at room temperature for 2 h, and washed 3 times with DPBS, 10 min for each time. After the staining with a DAPI staining solution for 1 min, the staining solution was discarded, and the cells were sealed with 50% glycerol and observed under a fluorescent microscope (FIG. 4).

As shown in FIG. 4, by immunofluorescence staining, a germ cell marker VASA (green) and an SSCs marker UCHL1 (red) could be obviously stained in the separated and cultured cells. Therefore, the separated and cultured cells were SSCs.

Currently, fresh testicular tissues are used for the separation and culture of bovine testicular tissues, and there is no reported method for separating and culturing SSCs using a frozen bovine testicular tissue. The present disclosure innovatively proposes a method for cryopreserving a bovine testicular tissue and culturing SSCs after resuscitation. In addition, the present disclosure further innovatively proposes a fragment culture method of bovine seminiferous tubules, such that the cells can naturally migrate to enrich and purify the SSCs. In the bovine SSCs separated and cultured by the method of the present disclosure, cluster-like proliferation of SSCs in the PI generation has been observed.

### Example 2

This example was carried out the same with Example 1 except that: in step (3), the collagenase IV solution had a concentration of 0.5 mg/mL, and the DNase I solution had a concentration of 0.5 mg/mL.

### Example 3

This example was carried out the same with Example 1 except that: in step (3), the collagenase IV solution had a concentration of 2 mg/mL, and the DNase I solution had a concentration of 2 mg/mL.

### Example 4

This example was carried out the same with Example 1 except that: the digestion was conducted in a water bath at 38° C. for 13 min.

### Example 5

This example was carried out the same with Example 1 except that: the digestion was conducted in a water bath at 36° ° C. for 16 min.

The above are merely preferred embodiments of the present disclosure. It should be noted that several improvements and modifications may further be made by a person of ordinary skill in the art without departing from the principle of the present disclosure, and such improvements and modifications should also be deemed as falling within the protection scope of the present disclosure.

What is claimed is:

1. A method for separating SSCs from a frozen testicular tissue, comprising the following steps:
resuscitating a frozen testicular tissue, digesting the resuscitated testicular tissue with a collagenase IV and a DNase I until seminiferous tubules are exposed and tube walls become coarse: washing the digested testicular tissue with a DPBS, conducting centrifugation and culture until cells migrate; collecting the migrated cells, and enriching and purifying SSCs.

2. The method according to claim 1, wherein the resuscitating comprises: resuscitating a cryopreservation tube in a water bath at 36° ° C. to 38° ° C. until the cryopreservation tube is completely thawed.

3. The method according to claim 1, wherein the frozen testicular tissue comprises a frozen bovine testicular tissue.

4. The method according to claim 1, wherein the resuscitated testicular tissue is digested by sequentially adding the DPBS, a collagenase IV solution, and a DNase I solution that are in an equal volume.

5. The method according to claim 1, wherein the collagenase IV solution has a concentration of 0.5 mg/mL to 2 mg/mL, and the DNase I solution has a concentration of 0.5 mg/mL to 2 mg/mL.

6. The method according to claim 1, wherein the digesting is conducted by incubation in a water bath at 36° ° C. to 38° C.

7. The method according to claim 1, wherein the culture is conducted with a DFS medium in an incubator at 36° ° C. to 38° C. with 5% CO₂.

8. The method according to claim 1, wherein enriching and purifying the SSCs comprises: conducting Percoll gradient centrifugation and purification in sequence to obtain the SSCs.

9. The method according to claim 8, wherein the Percoll gradient centrifugation comprises: adding a 40% Percoll solution, a 20% Percoll solution, and a suspension of the migrated cells sequentially into a centrifuge tube, conducting centrifugation, and collecting an interphase layer of the 40% Percoll solution and the 20% Percoll solution to obtain the SSCs.

10. The method according to claim 9, wherein the 40% Percoll solution, the 20% Percoll solution, and the suspension of the migrated cells are at a volume ratio of 1:1:2.
